(19) United States
(12) Patent Application Publication
(10) Pub. No.: US 2022/0061747 A1
(43) Pub. Date: Mar. 3, 2022

(54) APPARATUS AND METHOD FORA UTERINE THERMOCHROMIC PROBE

(71) Applicant: Joshua Huhndorf, Port Ludlow, WA (US)

(72) Inventor: Joshua Huhndorf, Port Ludlow, WA (US)

(21) Appl. No.: 17/240,598

(22) Filed: Apr. 26, 2021

Related U.S. Application Data
(60) Provisional application No. 63/071,577, filed on Aug. 28, 2020.

(51) Int. Cl. A61B 5/00; A61B 5/01; A61B 90/00

(52) U.S. Cl. CPC A61B 5/4325; A61B 2090/062; A61B 90/06; A61B 5/01

(57) ABSTRACT

A uterine thermochromic probe comprising a probe handle, a probe head, and a probe body comprising a thermochromic material, wherein the probe body couples the probe handle to the probe head. In some examples, the probe body tapers from a wide end at the probe handle to a narrow end at the probe head. In some further examples, the uterine thermochromic probe is formed from a continuous piece of thermochromic material.

### CROSS REFERENCE TO RELATED APPLICATIONS

This application claims priority to copending U.S. Application, Ser. No. 63/071,577, filed on Aug. 28, 2020, which is hereby incorporated by reference for all purposes.

### BACKGROUND

The present disclosure relates generally to various examination and diagnostic tools for examination of female human patients. In particular, examination of a female's cervix and/or uterus. For example, legacy uterus sounds are probe type instruments that are used to measure the length and direction of, and/or to dilate, the cervix and/or uterus of the female patient. As another example, legacy probes are to measure depth from body fluid markings on the probes or using additional non-sterile instruments to mark depth.

Known instruments and probes are not entirely satisfactory for the range of applications in which they are employed. Such uterine examinations, also referred to as uterine sounding, may require the use of multiple different sounds and probes. Use of multiple different sounds and/or probes may result in longer examination periods, discomfort to the female patients, and/or may even cause tissue damage. Accordingly, there is a need in the art to provide an improved apparatus and method to conduct examination of the cervix and/or uterus of a female patient

Thus, there exists a need for widgets that improve upon and advance the design of known widgets. Examples of new and useful widgets relevant to the needs existing in the field are discussed below.

### SUMMARY

The present disclosure is directed to a uterine thermochromic probe comprising a probe handle, a probe head, and a probe body comprising a thermochromic material, wherein the probe body couples the probe handle to the probe head. In some examples, the probe body tapers from a wide end at the probe handle to a narrow end at the probe head. In some further examples, the uterine thermochromic probe is formed from a continuous piece of thermochromic material.

### BRIEF DESCRIPTION OF THE DRAWINGS

FIG. 1 is a top view of a uterine thermochromic probe.

FIG. 2 is a bottom view of an embodiment of the uterine thermochromic probe.

FIG. 3 is an isometric view of the uterine thermochromic probe.

FIG. 4 is another isometric view of uterine thermochromic probe.

FIG. 5 is a close-up isometric view of the handle end of the uterine thermochromic probe.

FIG. 6 is an isometric view of a uterine thermochromic probe showing a color change.

FIG. 7 is a color photograph of a plurality of uterine thermochromic probes showing a color change occurring to one of the uterine thermochromic probes when placed in a warm glass of water.

### DETAILED DESCRIPTION

The disclosed uterine thermochromic probes will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various uterine thermochromic probes are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire-based connector, whether directly or indirectly through a communication network.

“Controllably coupled” means that an electronic device controls operation of another electronic device.

Apparatus and Method for a Uterine Thermochromic Probe

With reference to the figures, uterine thermochromic probes will now be described. The uterine thermochromic probes discussed herein function to provide information from an examination of a female's cervix and/or uterus. For example, the tactile sensations felt by the medical practitioner while using the uterine thermochromic probes allows the medical practitioner to sense various anatomical characteristics of the examined cervix and/or uterus. The uterine thermochromic probes color changes in response to the patient's body temperature, allowing the medical practitioner to more accurately measure a location of interest within the uterus of the female patient. The medical practitioner may visually see the location of the color transition on the uterine thermochromic probes after examination, and then visually determined a length and direction of the patient's cervix and/or uterus by inspecting measurement indicators on the uterine thermochromic probes. The uterine thermochromic probes may also be used to dilate patient's cervix and/or uterus.

The reader will appreciate from the figures and description below that the presently disclosed uterine thermochromic probes address many of the shortcomings of conventional uterine instruments and probes. For example, the uterine thermochromic probes provide information that only multiple conventional instruments and/or probes. Utilizing a single probe reduces examination periods, discomfort to the female patients, and/or may reduce tissue damage.

Uterine Thermochromic Probe

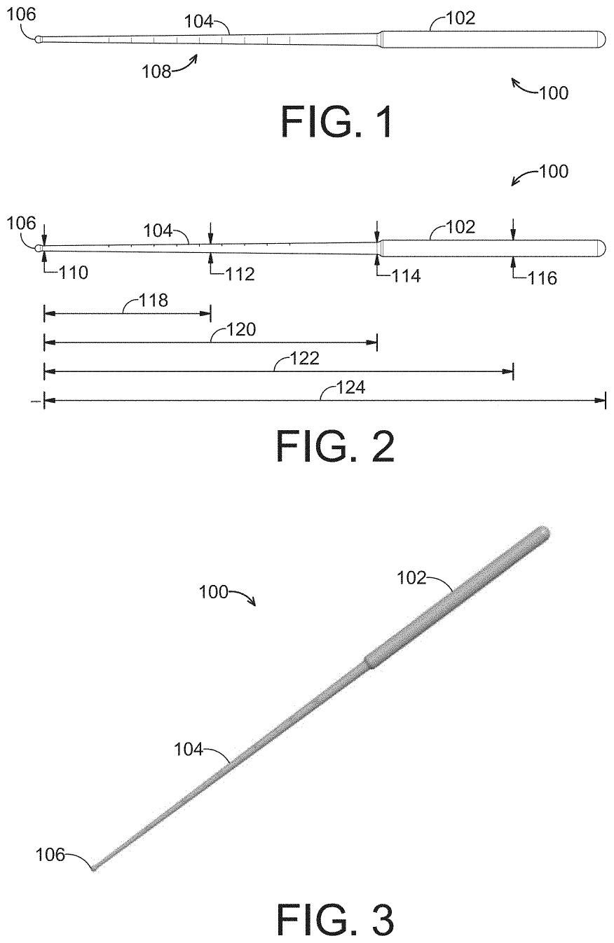

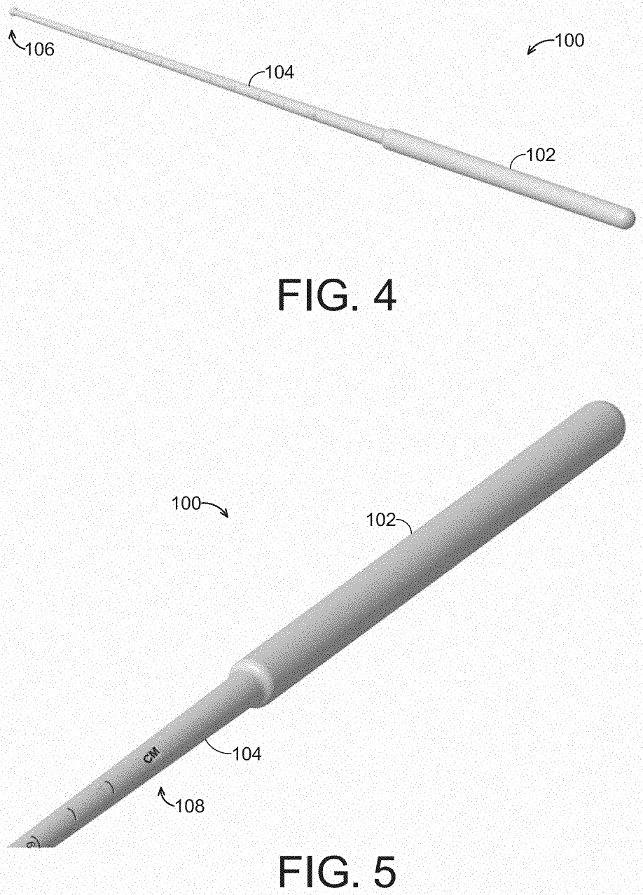

With reference to FIGS. 1-5, a first example of a uterine thermochromic probe, uterine thermochromic probe 100, will now be described. The uterine thermochromic probe 100, interchangeably referred to herein as a uterine thermochromic sound 100, comprises a probe handle 102, a probe body 104, and a probe head 106. A plurality of measurement indicators 108 are located on the probe body 104 that indicate distance from the probe head 106. Embodiments of the uterine thermochromic probe 100 are made of a color sensitive material, or employ a color sensitive material coating, on the probe body 104 that changes color in response to being exposed to a threshold temperature. The measurement indicators 108 may be presented in any suitable system of measurement, such as inches, centimeters, or the like. Fractional indicators may be used, such as a half inch, a quarter inch, millimeters, or the like.

The various embodiments of the uterine thermochromic probe 100 employ a thermochromic material that changes color when the temperature of the thermochromic material reaches or exceeds a threshold temperature. In a preferred embodiment, the threshold temperature is greater than the ambient air temperature typically encountered in an examination room (or a temperature that might be encountered while the uterine thermochromic probe 100 is in transport to the examination room). Preferably, the threshold temperature is less that the normal body temperature of the female patient who is undergoing an examination. In an example embodiment, the threshold temperature of the thermochromic material is approximately thirty-one degrees (31°) Celsius (87.8° Fahrenheit). Any suitable thermochromic material may be used in the various embodiments. Further, any suitable threshold temperature, or threshold temperature range, may be defined by the selected thermochromic material in the various embodiments. In an example embodiment, the threshold temperature range is thirty degrees (30°) to forty degrees (40°) Celsius.

In practice, the medical practitioner may have many uterine thermochromic probes 100 of different lengths and sizes to facilitate examination of different ages and/or sizes of female patients. Further, a plurality of different uterine thermochromic probes 100 may be used to measure different parts of the cervix and/or uterus of a particular female patient. Each uterine thermochromic probe 100, when withdrawn, accurately indicates the depth of the uterine thermochromic probe 100 into a particular part of the cervix and/or uterus of the female patient. After noting the insertion lengths based on visual inspection of the location of the color change and the measurement indicators 108, the uterine thermochromic probe 100 may be discarded into a suitable container.

In some embodiments, as shown in FIG. 2, the uterine thermochromic probe 100 may comprise a tapered shape to facilitate dilation of the cervix and/or uterus of a female patient. Dilation may be medically relevant at different lengths and/or depths of the uterus. In one example, the dimensions of the uterine thermochromic probe 100 are as follows. A length 124 of the uterine thermochromic probe 100 measures two hundred fifty (250) millimeters (mm). A first section 118 of the uterine thermochromic probe 100 is sixty (60) millimeters (mm) in length, as measured from a first diameter 110, where the probe head 108 and the probe base 104 meet, to a second diameter 112 located approximately mid-way on the probe body 104. The first section 118 increases in diameter from two (2) millimeters (mm) at the first diameter 110 to four and a half (4.5) millimeters (mm) at the second diameter 112. A second section 120 is eighty (80) millimeters (mm) in length, as measured from the first diameter 110 to a third diameter 114 located where the probe body 104 and the probe handle 102 meet. The second section 120 continues to increase in diameter from four and a half (4.5) millimeters (mm) at the second diameter 112 to six (6) millimeters (mm) at the third diameter 114. A third section 122 is one hundred fifty (150) millimeters (mm) in length, as measured from the first diameter 110 to a fourth diameter 116 located approximately mid-way on the probe handle 102. The third section 122 continues to increase in diameter from six (6) millimeters (mm) at the third diameter 114 to seven (7) millimeters (mm) at the fourth diameter 116. In another embodiment, the probe handle 102 does not taper and remains a consistent seven (7) millimeters (as shown in FIG. 2). There may be a small tapered transition near the third diameter 114 as a transition between the probe body 104 and the probe handle 102. The probe handle 102 may also have a cap at its end, such as a rounded cap as shown in FIG. 2.

In another example, a larger uterine thermochromic probe 100 comprises the following dimensions. The first section 118 increases in diameter from four (4) millimeters (mm) at the first diameter 110 to seven (7) millimeters (mm) at the second diameter 112. The second section 120 continues to increase in diameter from seven (7) millimeters (mm) at the second diameter 112 to nine (9) millimeters (mm) at the third diameter 114. The third section 122 continues to increase in diameter from nine (9) millimeters (mm) at the third diameter 114 to ten (10) millimeters (mm) at the fourth diameter 116.

In some examples, the uterine thermochromic probe may be sterilized and saved for re-use if the thermochromic material of the probe body 104 changes back to its original color. Alternatively, embodiments that employ a cover or strip of thermochromic material that is removably secured to the probe body 104 can be reused by removing the used thermochromic material, performing a sterilization of the uterine thermochromic probe 100, and then securing new thermochromic material to the uterine thermochromic probe 100.

Probe Handle 102

As can be seen in FIGS. 1-5, the probe handle 102 is wider in diameter than the probe body 104 and the probe head 106. The probe handle 102 is coupled to the probe body 104. As show in FIGS. 1-2, a small, tapered connection couples the probe handle 102 and the probe.

The medical practitioner conducting the examination will experience tactile sensations in the probe handle 102, which are communicated from the probe body 104 and/or the probe head 106 back to the probe handle 102. The tactile sensations felt by the medical practitioner allow the medical practitioner to sense various anatomical characteristics of the examined cervix and/or uterus of the patient.

Probe Body 104

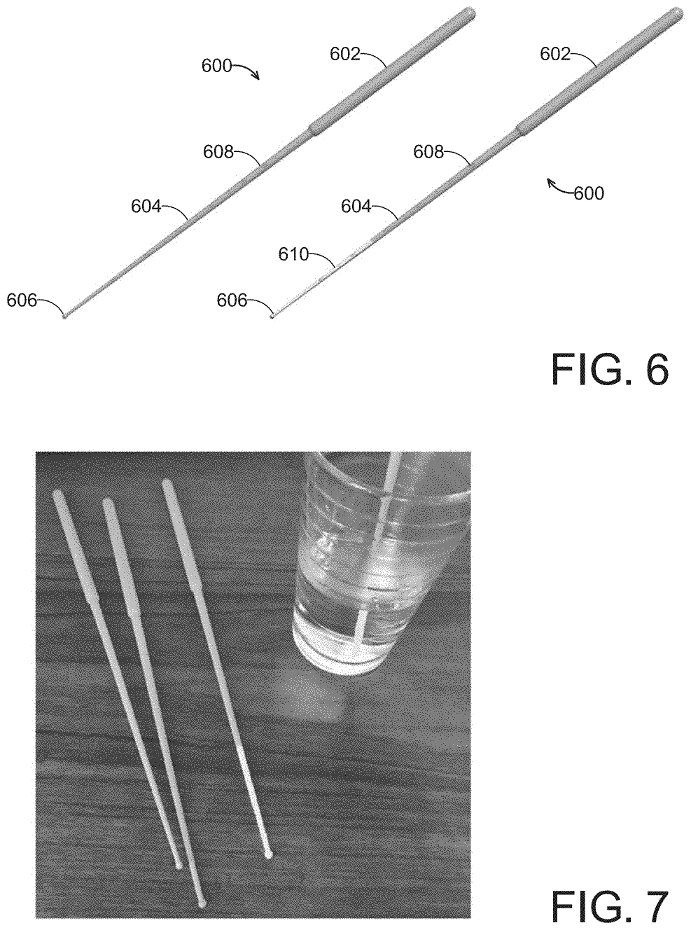

In the example shown in FIGS. 1 and 2, the probe body 104 couples the probe handle 102 to the probe head 106. The probe body 104 includes the measurement indicators 108. The probe body 104 is comprised of a thermochromic material. In an example embodiment, the thermochromic material is made of a leuco-dye mixture composed of a former color, a color developer, and a solvent. The former color determines the base color of the probe body 104 before use during the examination (See FIG. 6 and the corresponding description below). That is, the former color is the color of the probe body 104 before use.

The melting point of the solvent defines the threshold temperature at which the thermochromic material changes color. In response to the thermochromic material reaching or exceeding the threshold temperature, the solvent causes the color developer to change the color of the thermochromic material. Any suitable color may be used for the former color and the color developer so long as the difference between the colors are significantly different so as to enable the medical practitioner to visually identify the location of the transition between the former color and the color developer.

In other embodiments, other parts of, or all of, the uterine thermochromic probe 100 are made of the thermochromic material. In some embodiments, the probe body 104 is covered in a layer or coating of the thermochromic material. In some embodiments, a cover or strip of thermochromic material may be removably secured to the probe body 104 to facilitate later reuse of the uterine thermochromic probe 100.

Preferably, the probe body 104 is made of a semi-rigid, yet flexible, material. The flexible probe body 104 facilitates movement and passage of the probe head 106 of the uterine thermochromic probe 100 into and through the cervix and/or uterus of the female patient during an examination. The medical practitioner conducting the examination will experience tactile sensations in the probe handle 102, which are communicated from the probe body 104 and/or the probe head 106 back to the probe handle 102. The tactile sensations felt by the medical practitioner allows the medical practitioner to sense various anatomical characteristics of the examined cervix and/or uterus.

Probe Head 106

In the example shown in FIGS. 1-4, the probe head 106 is connected to the probe body 104. The shape of the probe head 106 is preferably round, or substantially round, to facilitate entry of the probe head 106 into the cervix and/or uterus, passage through the cervix and/or uterus, and then exit from the cervix and/or uterus of the female patient. Other probe head 106 shapes may be used in the various embodiments, such as a bulbous head as used in a brake sound or rosebud sound, a rounded end as used in a Dittel sound, a curved end as used in a Hank sound, or a pronounced tip as used in a Van Buren sound. Some embodiments may even have multiple extending portions for the probe body 104, such as in a Pratt sound.

The shape of the probe head 106 and the flexibility of the probe body 104 are designed to minimize the possibility of wall perforation and tissue damage in the cervix and uterus of the female patient, and to facilitate the tactile sensation experienced by the medical practitioner during an examination. Because the probe body 104 changes color in response to the patient's body temperature, the medical practitioner can more accurately measure a location of interest within the uterus of the female patient. The medical practitioner may visually see the location of the color transition on the probe body 104 after removal of the uterine thermochromic probe 100 from the uterus, and then visually determined the length by inspecting the measurement indicators 108 on the probe body 104 (See FIG. 6 and the corresponding description below).

Plurality of Measurement Indicators 108

In the example shown in FIG. 1, the plurality of measurement indicators 108 are located on the probe body 104 that indicate distance from the probe head 106. FIG. 5 is a close-up isometric view of the uterine thermochromic probe 100 showing the probe handle 102 and the measurement indicators 108 in more detail. The measurement indicators 108 may be presented in any suitable system of measurement, such as inches, centimeters, or the like. Fractional indicators may be used, such as a half inch, a quarter inch, millimeters, or the like.

Color Change

FIG. 6 is an isometric view of uterine thermochromic probes 600 demonstrating a color change. The left uterine thermochromic probe 600 has not been used for examination of a female patient. The right uterine thermochromic probe 600 has been used for examination of a female patient and indicates a color change, wherein the former color 610 is a dark gray color and the color developer 612 is a light gray color. The probe head 606 and a portion of the probe body 604 has transitioned to the light gray color of the color developer 612. The location of the transition between the former color 610 and the color developer 612 indicates the depth of insertion of the uterine thermochromic probe 600 into the female patient's uterus. The measurement indicators 608 enable the medical practitioner to visually identify the insertion length.

FIG. 7 is a color photograph of a plurality of uterine thermochromic probes 700 showing a color change occurring to one of the uterine thermochromic probes 700 when placed in a warm glass of water. Here, the temperature of the warm water is greater that the threshold temperature of the thermochromic material. The former color 710 is orange and the color developer 712 is yellow. Colors will vary depending on design. The probe head 706 and a portion of the probe body 704 that has been submerged in the warm water has transitioned to the yellow color of the color developer 712. The location of the transition between the former color 710 and the color developer 712 indicates the depth of insertion of the uterine thermochromic probe 700 into the female patient's uterus. The measurement indicators 708 enable the medical practitioner to visually identify the insertion length. One skilled in the arts appreciates that the other three uterine thermochromic probes 700 shown in FIG. 7, which are all colored orange of the former color 710, have not been used for examination of a female patient.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A uterine thermochromic probe, comprising:
a probe handle;
a probe head; and
a probe body comprising a thermochromic material, wherein the probe body couples the probe handle to the probe head.

2. The uterine thermochromic probe of claim 1, further comprising measurement indicators on the probe body.

3. The uterine thermochromic probe of claim 3, wherein the measurement indicators indicate a distance from the probe head.

4. The uterine thermochromic probe of claim 1, wherein the thermochromic material comprises a threshold temperature range.

5. The uterine thermochromic probe of claim 5, wherein the threshold temperature range is thirty to forty degrees Celsius.

6. The uterine thermochromic probe of claim 5, wherein the thermochromic material changes color when the threshold temperature is reached.

7. The uterine thermochromic probe of claim 1, wherein the probe body tapers from the probe handle to the probe head.

8. The uterine thermochromic probe of claim 1, wherein the thermochromic material is removeable.

9. A uterine thermochromic probe, comprising:
a probe handle;
a probe head; and
a probe body comprising a thermochromic material, where the probe body couples the probe handle to the probe head, and wherein the probe body tapers from a wide end at the probe handle to a narrow end at the probe head.

10. The uterine thermochromic probe of claim 9, wherein the wide end measures 7 mm in diameter to facilitate dilation.

11. The uterine thermochromic probe of claim 9, wherein the narrow end measures 2 mm in diameter.

12. The uterine thermochromic probe of claim 9, further comprising measurement indicators on the probe body.

13. The uterine thermochromic probe of claim 12, wherein the measurement indicators indicate distance from the probe head.

14. The uterine thermochromic probe of claim 9, wherein the thermochromic material comprises a threshold temperature range.

15. The uterine thermochromic probe of claim 14, wherein the threshold temperature range is thirty to forty degrees Celsius.

16. A uterine thermochromic probe, comprising:
a probe handle;
a probe head;
a probe body coupling the probe handle to the probe head; and
wherein the uterine thermochromic probe is formed from a continuous piece of thermochromic material.

17. The uterine thermochromic probe of claim 16, further comprising measurement indicators on the probe body.

18. The uterine thermochromic probe of claim 17, wherein the measurement indicators indicate distance from the probe head.

19. The uterine thermochromic probe of claim 16, wherein the thermochromic material comprises a threshold temperature range.

20. The uterine thermochromic probe of claim 16, wherein the threshold temperature range is thirty to forty degrees Celsius.
